(19) United States
(12) Patent Application Publication
(10) Pub. No.: US 2004/0029871 A1
(43) Pub. Date: Feb. 12, 2004

(54) Pharmaceutical compounds for treating copd

(76) Inventors: Kam-Wah Thong, Loughborough (GB); Anthony J Kettle, Christchurch (NZ)

Correspondence Address: Janis K Fraser, Fish & Richardson, 225 Franklin Street, Boston, MA, 02110-2804, US

(21) Appl. No.: 10/275,824

(22) PCT Filed: May 8, 2001
(86) PCT No.: PCT/SE01/01014

(30) Foreign Application Priority Data
May 12, 2000 (GB) 0011358.9

(51) Int. Cl. A61K 31/549; A61K 31/4965; A61K 31/513; A61K 31/405; A61K 31/426; A61K 31/11; A61K 31/135

(52) U.S. Cl. 514/222.8; 514/269; 514/365; 514/419; 514/345; 514/255.06; 514/646; 514/699

(57) ABSTRACT

Use of an MPO inhibitor for the treatment of COPD.

The present invention relates to the use of certain pharmaceutical compounds for the treatment of COPD.

COPD is a major cause of morbidity and mortality. A key etiological factor is smoking. It is apparent that smokers have elevated levels of MPO (Dash et al., Blood., 1991, 72, 1619; Bridges et al., Eur. J. Respir. Dis., 1985, 67, 84). Furthermore, there is circumstantial evidence to link MPO levels with the severity of lung disease in human subjects (Hill et al., Am. J. Respir. Crit. Care., 1999, 160, 893; Keatings & Barnes., Am. J. Respir. Crit. Care., 1997, 155, 449; Regelmann et al., Pediatric. Pulmonol., 1995, 19, 1; Linden et al., Am Rev. respir. Dis., 1993, 148, 1226). Myeloperoxidase is a heme protein that plays a vital role in the generation of toxic hypochlorus acid and free radicals, which may be involved in cellular damage and inflammation (Kettle & Winterbourn., Curr. Opin. Hematol., 2000, 7, 53). This protein has been implicated in a variety of different conditions (Klebanoff., Proc. Assoc. Am. Physicians., 1999, 111, 383). Compounds having activity as inhibitors of MPO are known in the art (Kettle & Winterbourn., Biochem. Pharmacol., 1991, 41, 10; Bozeman et al., Biochem. Pharmacol., 1992, 44, 553). For example, the compound dapsone, which is known to be an inhibitor of MPO has been linked to the treatment of various conditions, including a general reference to inflammatory diseases such as asthma (Berlow et al., J. Allergy Clin. Immunol., 1991, 87, 710). Interestingly, there is no specific mention of any synthetically derived chemical inhibitors of MPO being use for the treatment of COPD.

Current drugs used for treating COPD are not all fully effective. The need for novel and better drugs is essential to cope with the rising incidence of COPD (Peleman et al., Curr. Opin. Cardiovas. Pulmonary. Renal. Invest. Drugs., 1999, 1, 491). It has now surprisingly been found that compounds having activity as inhibitors of MPO are expected to be of potential use in the treatment of COPD.

In a first aspect the invention therefore provides the use of an MPO inhibitor for the treatment of COPD. It will be understood that the MPO inhibitors of the invention can be used therapeutically or as prophylactics.

Particularly suitable compounds include MPO inhibitors known in the art.

Preferred compounds include those listed below:

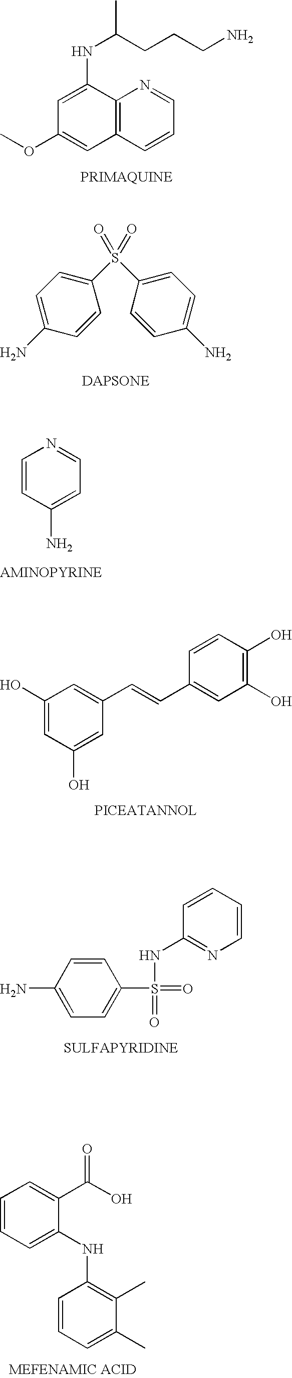

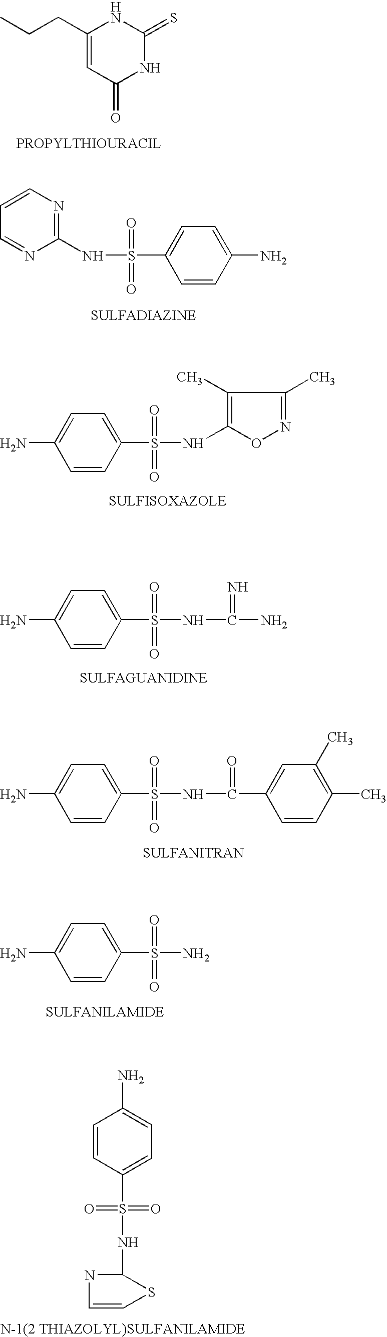

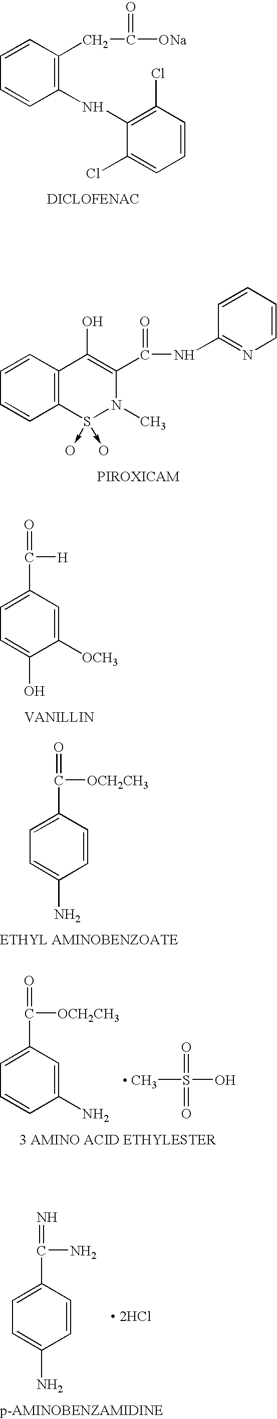

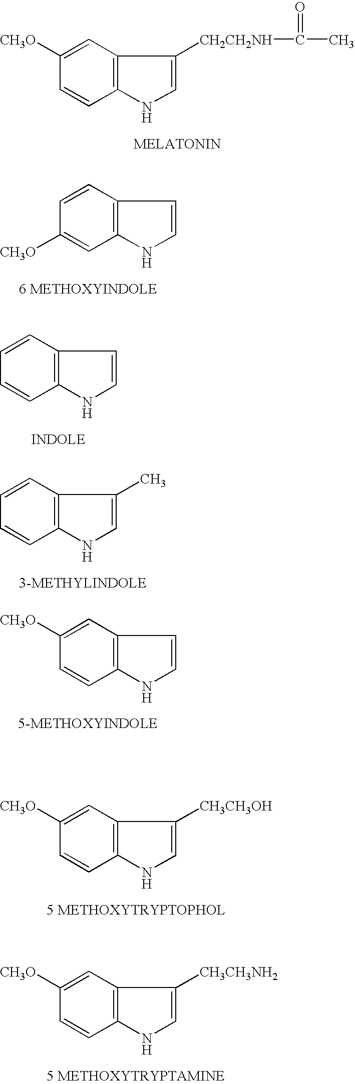

Additional preferred compounds include the following:

ISONIAZID

NITECAPONE

5-AMINOSALICYLIC ACID

PHENYLHYDRAZINE

D-PENICILLAMINE

TIOPRONIN

RESORCINOL

QUERCETIN

RUTIN

QUINACRINE

BAKUCHIOL

The above compounds can be used both as free bases and pharmaceutically acceptable salts. Suitable salts include all known pharmaceutically acceptable salts such as acid addition salts such as hydrochloride and malate salts.

Preferred compounds include primaquine, sulfanilamide, dapsone and sulfanilamide, in particular dapsone.

The invention also provides a method of treating or preventing COPD, which comprises administering to a patient an MPO inhibitor or a pharmaceutically acceptable salt thereof in particular by administering primaquine, dapsone, aminopyrine, piceatannol, mefenamic acid, sulfapyridine, sulfanilamide, propylthiouracil, sulfadiazine, sulfisoxazole, sulfaguanidine, sulfanitran, sulfanilamide, N-1(2-thiazolyl)sulfanilamide, diclofenac, piroxicam, vanillin, ethyl aminobenzoate, 3-aminoacid ethylester, p-aminobenzamide, melatonin, 6-methoxyindole, indole, 3-methylindole, 5-methoxyindiole, 5-methoxytryptophol, methoxytryptophol and pharmaceutically acceptable salts thereof.

In a further aspect the invention provides an MPO inhibitor, in particular a compound named above, in the manufacture of a medicament for use in the prevention or treatment of COPD.

Suitable daily dose ranges are from about 0.1 mg/kg to about 100 mg/kg. Unit doses may be administered conventionally once or more than once a day, for example, 2, 3, or 4 times a day, more usually 1 or 2 times a day. A typical dosing regime for dapsone or propylthiouracil would be oral once or twice a day at 100 mg or 300 mg, respectively.

The pharmaceutical composition comprising the MPO inhibitor of the invention may conveniently be in the form of tablets, pills, capsules, syrups, powders or granules for oral administration; sterile parental or subcutaneous solutions, suspensions for parental administration of suppositories for rectal administration, all of which are well known in the art.

The following examples illustrate the invention.

### EXAMPLE 1

Here we describe an in vitro MPO assay that was developed to assess inhibition of enzyme activity. Essentially the MPO assay was designed to measure the production of hypochlorus acid (HOCl), which is the key physiological product generated by the enzyme in vivo. An outline of the assay reactions is given:

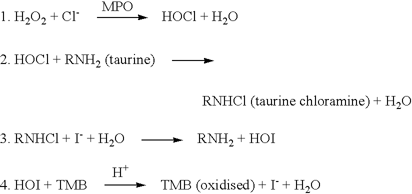

The reaction mixtures in 20 mM phosphate buffer pH 6.5 contained 2.5 nM MPO (purified human enzyme from Planta), 100 uM H₂O₂, 140 mM NaCl, 10 mM taurine, 20 uM tyrosine and compound solvent, DMSO, at 1%. Compounds were preincubated with the MPO enzyme in buffer for about 15 min prior to start of reaction with H₂O₂. The whole reaction was carried out at room temperatutre for 10 min in a 96-well plate. The reaction was terminated by a stop/developing reagent, which consist in their final concentration of Glacial acetic acid (400 mM), KI (100 uM) and TMB in dimethylformamide (10 mM). All test concentrations were done in duplicated with at least two separate determinations n=2, unless otherwise stated. The inhibitory concentration for a compound is presentated as pIC50, which is −log IC50.

Various compounds have been tested against the human MPO. It can be seen that dapsone is the most potent inhibitor of the sulfones/sulfonamides tested. Indoles and other compounds are also effective in blocking the production of HOCl by human MPO. All data obtained for the sulfones/sulfonamides, indoles and miscellaneous are presented in Table 1, 2 and 3, respectively.

TABLE 1
Inhibtion of human MPO-HOCl production by
sulfones/sulfonamides

| Compound | pIC50 |
|---|---|
| Dapsone | 6.2 |
| N-1(2 thiazolyl)-sulfanilamide | 6.0 |
| Sulfanilamide | 6.0 |
| Sulfapyridine | 5.7 |
| Sulfaguanidine | 5.5 |
| Sulfisoxazole | 5.2 |
| Sulfadiazine | 5.2 |
| Sulfanitran | 5.1 |

TABLE 2
Inhibition of human MPO-HOCl production by indoles

| Compound | pIC50 |
|---|---|
| 5-Methoxytryptophol | 6.3 |
| 5-Methoxytryptamine | 6.2 |
| Melatonin | 6.1 |
| 3-Methylindole | 5.9 |
| 6-Methoxyindole | 5.8 |
| Indole | 5.7 |
| 5-Methoxyindole | 5.6 |

TABLE 3
Inhibition of human MPO-HOCl production

| Compound | pIC50 |
|---|---|
| Ethyl aminobenzoate | 6.2 |
| 3-Aminobenzoic acid ethylester | 6.2 |
| p-Aminobenzamidine | 5.6 |
| Piroxicam | 5.6 (n = 1) |
| Diclofenac | 5.4 |
| Vanillin | 5.1 |

### EXAMPLE 2

Here we describe the use of a functional human neutrophil assay to determine the effects of MPO inhibitors on the production of HOCl. This assay detects the production of HOCl from stimulated (e.g. PMA, LPS, fMLP, zymozan) human neutrophils. Human neutrophils were purified from fresh heparinised blood by density centrifugation on Polymorphprep (Nycomed). These neutrophils were used immediately after purification. A standard reaction mixture contained the following: 2×106 neutrophils, 140 mM NaCl, 5 mM taurine, 0.5 mM MgCl₂, 1 mM CaCl₂ and 1 mg/ml glucose. Test compounds were made up in DMSO and added to cells, with a final DMSO concentration of 0.5%. Test compounds were given 15 min preincubation at 37C with neutrophils prior to the addition of the PMA stimulant (1 μg/ml). The assay was then allowed to progress for another 30 min at 37C. At the end of the incubation, supernatants were collected by centrifugation and assayed for HOCl by using the stop/development reagent as above. All compounds were tested in duplicate with at least two separate determinations n=2 from two different donors.

The data for some of these inhibitors are shown in Table 4.

TABLE 4
Inhibition of HOCl production by stimulated human neutrophils

| HOCl production by neutrophils | pIC50 |
|---|---|
| Primaquine | 4.9 |
| Sufanilamide | 4.8 |
| Dapsone | 4.7 |
| Sulfapyridine | 4.5 |

We have also shown that under the assay conditions and concentrations of inhibitors used, human neutrophils were not affected by cytotoxicity, as assessed by the release of lactate dehydrogenase from damaged neutrophils. Lactate dehydrogenase activity was measured as described by Boehringer Mannheim GmbH, Sandhofer Strabe 116, D-68305 Mannheim, Germany (Cytotoxicity Detection Kit-LDH-Cat No: 1 644 793).

### EXAMPLE 3

There are several animal models of COPD, which can be employed for the testing of MPO inhibitors. These models have been referred in the reviews of Snider (Chest., 1992, 101, 74S) and Shapiro (Am. J. Respir. Cell Mol. Biol., 2000, 22, 4). In our study, we prefer the LPS- and/or smoking-induced lung injury rodent model. Mice or rats can be be dosed (by any of the following routes: ip, po, iv, sc or aerosol) with MPO inhibitors prior to LPS and/or smoking challenge. After an appropriate set interval, the animals are sacrificed and assessed for lung injury (similar to the work reported by Faffe et al., Eur. Respir. J., 2000, 15, 85; Suntres & Shek., Biochem. Pharmacol., 2000, 59, 1155; Vanhelden et al., Exp. Lung. Res., 1997, 23, 297). MPO activity of the lung lavage fluids (BAL), lung tissues, neutrophils and whole blood are then measured. Blood samples can be analysed for inflammatory cells and cytokines (e.g. TNFα). Histology and biochemical markers (e.g. chlorinated protein, lactate dehydrogenase, alkaline phosphatase) for lung cellular damage can be assessed. The efficacies of the MPO inhibitors are measured against their abilities to reduce/prevent lung injury. It is expected these MPO inhibitors will be therapeutically or prohylactically effective in these models.

1. Use of an MPO inhibitor for the treatment of COPD.

2. Use according to claim 1 where the compound having MPO inhibitory activity is primaquine, dapsone, aminopyrine, piceatannol, mefenamic acid, sulfapyridine, sulfanilamide, propylthiouracil, sulfadiazine, sulfisoxazole, sulfaguanidine, sulfanitran, sulfanilamide, N-1(2-thiazolyl)sulfanilamide, diclofenac, piroxicam, vanillin, ethyl aminobenzoate, 3-aminoacid ethylester, p-aminobenzamide, melatonin, 6-methoxyindole, indole, 3-methylindole, 5-methoxyindiole, 5-methoxytryptophol, 5-methoxytryptamine and pharmaceutically acceptable salts thereof.

3. A method of treating or preventing COPD in a mammal which comprises administering a compound having MPO inhibiting activity or a pharmaceutically acceptable salt thereof.

4. A method according to claim 3 in which the MPO inhibitor is selected from primaquine, dapsone, aminopyrine, piceatannol, mefenamic acid, sulfapyridine, sulfanilamide, propylthiouracil, sulfadiazine, sulfisoxazole, sulfaguanidine, sulfanitran, sulfanilamide, N-1(2-thiazolyl)sulfanilamide, diclofenac, piroxicam, vanillin, ethyl aminobenzoate, 3-aminoacid ethylester, p-aminobenzamide, melatonin, 6-methoxyindole, indole, 3-methylindole, 5-methoxyindiole, 5-methoxytryptophol, 5-methoxytryptamine and pharmaceutically acceptable salts thereof.

5. Use of an MPO inhibitor in the manufacture of a medicament for use in the prevention or treatment of COPD.

6. Use according to claim 7 in which the MPO inhibitor is selected from primaquine, dapsone, aminopyrine, piceatannol, mefenamic acid, sulfapyridine, sulfanilamide, propylthiouracil, sulfadiazine, sulfisoxazole, sulfaguanidine, sulfanitran, sulfanilamide, N-1(2-thiazolyl)sulfanilamide, diclofenac, piroxicam, vanillin, ethyl aminobenzoate, 3-aminoacid ethylester, p-aminobenzamide, melatonin, 6-methoxyindole, indole, 3-methylindole, 5-methoxyindiole, 5-methoxytryptophol, 5-methoxytryptamine and pharmaceutically acceptable salts thereof.
